(19) United States
(12) Patent Application Publication
(10) Pub. No.: US 2022/0061959 A1
(43) Pub. Date: Mar. 3, 2022

(54) VOICE-ASSISTED DENTAL SYSTEMS

(71) Applicant: 3M INNOVATIVE PROPERTIES COMPANY, St. Paul, MN (US)

(72) Inventors: Nitsan Ben-Gal Nguyen, Apple Valley, MN (US); Guruprasad Somasundaram, Woodbury, MN (US); Alexandra R. Cunliffe, St. Paul, MN (US); Vera Shuman, St. Paul, MN (US); Elisa J. Collins, St. Paul, MN (US)

(21) Appl. No.: 17/416,818

(22) PCT Filed: Dec. 23, 2019
(86) PCT No.: PCT/IB2019/061318

§ 371 Date: Jun. 21, 2021

Related U.S. Application Data
(60) Provisional application No. 62/785,607, filed on Dec. 27, 2018.

(51) Int. Cl. A61C 7/00; G10L 15/18

(52) U.S. Cl. CPC A61C 7/002; G10L 15/1822

(57) ABSTRACT

Aspects of the present disclosure relate to methods, devices, and systems using speech recognition in a dental procedure. The method can include receiving, by a computing device, voice input data that specifies dental information for a patient. The method can also include analyzing, by the computing device, the voice input data to process the dental information. Based on the identified dental treatment information, the method can include generating, by the computing device, one or more custom dental prescriptions for the patient.

### TECHNICAL FIELD

This disclosure is directed to computing systems that process dental (e.g., orthodontic) information, and that, in some examples, process voice inputs to assist in the processing of the dental information.

### BACKGROUND

The goal of the orthodontic treatment planning process is to determine where the post-treatment positions of a person's teeth (setup state) should be, given the pre-treatment positions of the teeth in a malocclusion state. Orthodontic treatments are typically administered manually, with the assistance of interactive computing systems as planning and modeling tools.

### SUMMARY

This disclosure generally describes systems that leverage speech recognition technology in evaluating the orthodontic state of a patient and/or for generating orthodontic treatment information for the patient. While this disclosure describes numerous examples with reference to orthodontic treatment, the systems of this disclosure may implement the described techniques with respect to other types of dental treatments, as well. In various examples, the systems of this disclosure use voice input processing, such as natural language processing (NLP), to interpret a clinician's spoken commands and generate orthodontic treatment information therefrom. The systems of this disclosure may also provide various types of output, including, but not limited to, projected realignments of groups of teeth, comparative data between a patient's present dental state and one or more previous dental states, dental scan information, etc.

In one example, this disclosure is directed to a method that includes receiving, by a computing device, voice input data that specifies dental information for a patient, analyzing, by the computing device, the voice input data to process the dental information, and based on the identified dental treatment information, generating, by the computing device, one or more custom dental prescriptions for the patient.

In another example, this disclosure is directed to a method that includes receiving, by a computing device, voice input data specifying a patient and information for modifying a configuration of a tooth or a group of teeth of the patient, identifying, by the computing device and based on the voice input data, stored data associated with the tooth or group of teeth of a patient, and generating, by the computing device, based on the voice input and the stored data, one or more dental arrangements in which the configuration of the group of teeth is modified according to the information for modifying the configuration.

In another example, this disclosure is directed to a method that includes receiving, by a computing device, voice input data specifying a patient and dental scan information associated with the patient, and responsive to receiving the voice input, loading, by the computing device, the dental scan associated with the patient specified in the voice input data.

In another example, this disclosure is directed to a method that includes receiving, by a computing device, voice input data specifying at least information identifying a patient and a command, identifying, by the computing device and based on the command, a predetermined dental treatment setup, and assigning, by the computing device, the identified predetermined dental treatment setup to the patient.

In another example, this disclosure is directed to a method that includes receiving, by a computing device, voice input data specifying at least information identifying a patient and a command, identifying, by the computing device and based on the command, dental state information associated with the patient, and outputting, by the computing device, the dental state information associated with the patient.

In another example, this disclosure is directed to a method that includes receiving, by a computing device, voice input data specifying at least information identifying a patient and a command, comparing, by the computing device and based on the command, dental state information associated with the patient against dental state information for a population of patients to obtain comparative dental state information associated with the patient, and outputting, by the computing device, the comparative dental state information associated with the patient.

In another example, this disclosure is directed to a method that includes receiving, by a computing device, voice input data specifying at least information identifying a patient and a command, and comparing, by the computing device and based on the voice input data, a current dental state of the patient to a prior dental state of the patient.

In another example, this disclosure is directed to a method that includes receiving, by a computing device, voice input data, and selecting, based on data of the voice input data, by the computing device, a numbering system from a plurality of available numbering systems to be used for assessing a dental state of a patient. A numbering system as used herein represents a dental numbering system or dental notation that is used to identify individual teeth or a group of teeth. A numbering system as used herein may use a number-based nomenclature or a nomenclature that uses numbers in combination with letters to identify a tooth or a group of teeth.

The systems of this disclosure provide various technical improvements over existing technologies deployed at dental practices. As one example, by leveraging speech recognition, the systems of this disclosure enable a clinician to continue using his/her hands to inspect or treat the patient, while concurrently providing or obtaining information that is pertinent to evaluation and treatment. As another example, the systems of this disclosure potentially reduce data errors and/or incompleteness that could be caused by requiring a clinician to interrupt treatment/evaluation to then, with some delay, enter data using his/her hands. As another example still, by enabling on-the-fly entry of orthodontic treatment information during evaluation of a patient, the systems of this disclosure incentivize error correction by providing clinicians a second chance to review the original treatment proposal later.

The details of one or more examples of the techniques are set forth in the accompanying drawings and the description below. Other features, objects, and advantages of the techniques will be apparent from the description, drawings, and from the claims.

### BRIEF DESCRIPTION OF THE DRAWINGS

FIG. 1 is a conceptual diagram illustrating a system for voice input-based automated generation of orthodontic treatment information according to one or more aspects of this disclosure.

FIG. 2 is a conceptual diagram illustrating dental-oriented natural language processing aspects of this disclosure.

FIG. 3 is a conceptual diagram illustrating an example workflow of disclosure.

FIG. 4 is a block diagram illustrating an example implementation of the system illustrated in FIG. 1.

FIG. 5 is a block diagram illustrating another example implementation of the system illustrated in FIG. 1.

FIG. 6 is a conceptual diagram illustrating an example workflow of this disclosure.

FIG. 7 is a conceptual diagram illustrating an example workflow of this disclosure.

FIG. 8 is a conceptual diagram illustrating an example workflow of this disclosure.

FIG. 9 is a conceptual diagram illustrating an example workflow of this disclosure.

### DETAILED DESCRIPTION

Computing systems have been used to achieve final positions of teeth that are aesthetically and functionally correct according to best practices and orthodontists' opinions. Setup technicians and/or orthodontists can use orthodontic rules, for example, when determining the desired final positions of one or more teeth of a patient. These rules can be captured using metrics such as overbite, overjet, leveling, alignment, and others. By defining acceptable or desired levels of these various metrics, the systems can model what is a good setup. Acceptable or desired levels can be set through a combination of domain knowledge and thresholds inferred from data of historical final setups, or by data inputs provided by a clinician (e.g., dentist, orthodontist, technician, hygienist, etc.).

Teeth can be modeled as objects in three-dimensional (3D) space that have movement restrictions for certain types of motion. By converting dental positioning information into objective metrics, the problem of finding final setups can be formulated as an optimization problem where the goal is to get as many of the orthodontic metrics to acceptable levels as possible while significantly satisfying tooth motion limits. In general, the goal of orthodontic modeling and treatment is to achieve the best possible end state using these metrics without exceeding limits on arch form changes, extractions, crown movement, root movement, and IPR (interproximal reduction, also known as teeth shaving).

Systems of this disclosure enable clinicians to provide voice input (spoken observations, analysis, or instructions) to generate final setups, such as treatment plans, prescriptions, etc. As such, this disclosure sets forth systems and techniques for speech recognition as an input medium to improve the generation of final setups in dental and orthodontic treatment.

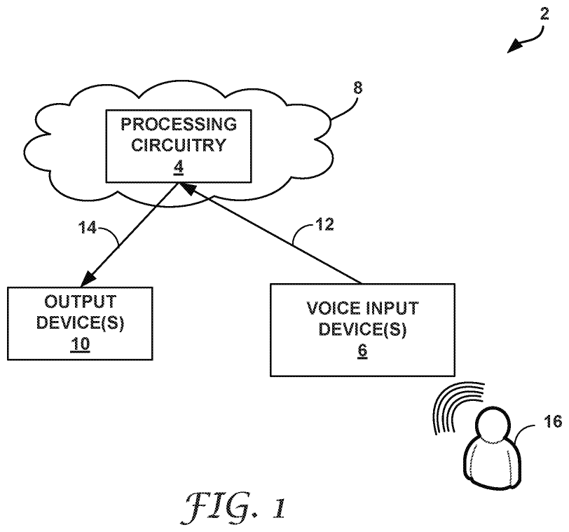

FIG. 1 is a conceptual diagram illustrating a system 2 for voice input-based automated generation of orthodontic treatment information according to one or more aspects of this disclosure. While system 2 is shown as a distributed (e.g., cloud-based) implementation in FIG. 1, in some examples, system 2 may also be a self-contained system that does not need to leverage cloud-based communication functionalities.

System 2 includes processing circuitry 4, one or more voice input devices 6, and one or more output devices 10. In the example of FIG. 1, processing circuitry 4 is coupled to voice input devices 6 and to output devices 10 via network 8. In various examples, processing circuitry 4 may include, be, or be part of programmable processing circuitry, fixed function circuitry, one or more processors, such as one or more digital signal processors (DSPs), general purpose microprocessors, application specific integrated circuits (ASICs), field programmable gate arrays (FPGAs), or other equivalent integrated or discrete logic circuitry, as well as any combination of such components. Network 8 may, in various examples, represent or include a private network associated with a business (e.g., a dental practice, a hospital, etc.) or other entity that has an interest in the location at which one or both of voice input devices 6 and/or output devices 10 are deployed. In other examples, network 8 may represent or include a public network, such as the Internet. Although illustrated as a single entity in FIG. 1 as an example, network 8 may include a combination of multiple public and/or private networks. For instance, network 8 may represent a private network implemented using public network infrastructure, such as virtual private network (VPN) tunnel implemented over the Internet. As such, network 8 may comprise one or more of a wide area network (WAN) (e.g., the Internet), a local area network (LAN), a VPN, and/or another wired or wireless communication network.

Voice input devices 6 may include various input or input/output (I/O) devices that can accept, relay, and optionally, process spoken inputs. Examples of voice input devices 6 include smartphones (e.g., iPhone® devices that implement Siri®, Android® devices, or Windows® phone equipment, etc.), tablet computers, assistant devices (e.g., Google® Home Mini, Amazon® Echo or other devices that leverage the Alexa® platform, etc.), or any other device that incorporates voice input capabilities, and in some cases, that incorporates some level of speech recognition capabilities. As such, voice input devices 6 may include devices that incorporate, or are communicatively coupled to, various types of microphones, such as single or arrayed microphones configured to capture audio data or a combination of audio data and descriptive metadata (as in the case of an EigenMike® microphone), or in some cases, may represent standalone implementations of these devices.

Voice input devices 6 are configured to receive spoken commands and input from clinician 16, who may represent a dentist, an orthodontist, a technician, a hygienist, an assistant, or any grouping of such individuals. Clinician 16 may provide the spoken commands/input while examining or treating a dental patient, or at other instances of time. As a non-limiting example, this disclosure discusses scenarios in which clinician 16 is actively working with a patient, also referred to herein as a “patient encounter.” During the course of a patient encounter, clinician 16 may provide various types of dental information, such as a dental evaluation of the patient, an orthodontic prescription, treatment plans that supplement or partially overlap with the orthodontic prescription, projections of single tooth or multi-tooth movements within a treatment plan or prescriptions, general observations on the patient's dental health, etc.

Voice input devices 6 may relay spoken input data 12 to processing circuitry 4 over network 8. Again, in various examples, and depending on the specific capability set or current configuration of voice input devices 6, spoken input data 12 may represent raw data or data that has been preprocessed to some extent. Processing circuitry 4 may parse and interpret spoken input data 12 to generate dental information related to a patient, such as the patient being examined/treated at the time of the patient encounter during which voice input devices 6 received the data pertaining to spoken input data 12. In various examples, processing circuitry 4 may process spoken input data 12 to generate different types of outputs, such as a final setup, an intermediate setup, a recommended course of action for the patient, a prescription, etc. Processing circuitry 4 may implement various types of speech input processing, such as NLP technology, to generate such outputs using spoken input data 12.

In turn, processing circuitry 4 may communicate dental information 14 to output devices 10 over network 8. Dental information 14 may include or represent portions of the information that processing circuitry 4 extracts or generates from spoken input data 12. As examples, dental information 14 may represent an intermediate or final orthodontia setup, a projected movement of a subset of the patient's teeth, a prescription, or corrective/critical feedback on the clinician's input as represented by spoken input data 12. In some examples, processing circuitry 4 may generate dental information 14 to include multiple treatment planning options for a single patient. For instance, processing circuitry 4 may include multiple different versions of a treatment plan, from which clinician 16 can select, based on various patient consultations or other criteria.

According to various examples of this disclosure, processing circuitry 4 may analyze and apply spoken input data 12 to run individual tooth movements/motions separately, or to run synergistic multi-tooth movements/motions concurrently. As such, processing circuitry 4 is configured to use spoken input data 12 to generate patient-facing or dentist-facing demonstrations of dental treatment options at varying levels of granularity. In this way, processing circuitry 4 may use various commands included in spoken input data 12 to generate multiple individual packages as treatment alternatives, or to generate multiple combinable sub-packages for an overall treatment plan.

In this way, according to some aspects of this disclosure, processing circuitry 4 may receive voice input data specifying a patient and information for modifying a configuration of a tooth or a group of teeth of the patient, identify, based on the voice input data, stored data associated with the tooth or group of teeth of a patient, and generate, based on the voice input and the stored data, one or more dental arrangements in which the configuration of the group of teeth is modified according to the information for modifying the configuration. In some examples, processing circuitry 4 may output a visual representation of the original dental arrangement and any of the one or more dental arrangements in which the tooth or group of teeth is moved. In some examples, processing circuitry 4 may output, for display, a visual representation of the original dental arrangement

According to some examples, to receive the voice input, processing circuitry 4 receives the voice input from a computer-mediated reality interface, and to output the visual representation of the prospective dental scan, processing circuitry 4 outputs the visual representation of the prospective dental scan to display hardware of the computer-mediated reality interface. In some examples the computer-mediated reality interface may include one of a VR or an AR interface. In some instances, the group of teeth includes two or more teeth, and wherein generating the prospective dental scan comprises generating data representing a synergistic movement of all of the two or more teeth included in the group.

In some examples, processing circuitry 4 may analyze the voice input data using natural language processing (NLP) to obtain information for modifying a configuration of a group of teeth, at least in part by identifying a dental dictionary, where the dental dictionary represents a subset of a general NLP dictionary, parsing the voice input data to obtain terms, and comparing one or more of the terms obtained from the parsed voice input data to a portion of the dental dictionary. In some examples, processing circuitry 4 may determine that at least one of the terms obtained from the parsed voice input does not match any entry of the dental dictionary and may discard the at least one term that does not match any entry of the dental dictionary without using the at least one term in generating the one or more dental arrangements.

Output devices 10 may include a variety of devices capable of presenting data that is intelligible to a human recipient. Examples of output devices 10 include display devices (e.g., monitors, televisions, tablet computer screens, smartphone screens, etc.), speakers (e.g. dedicated loudspeakers, headphones, in-built speakers of tablet computers or smartphones, etc.) haptic feedback devices, virtual reality (VR) or augmented reality (AR) headsets or environments, and others. Output devices 10 may be deployed at the dental office from which clinician 16 provided the voice commands that resulted in spoken input data 12, at another location administrated by staff associated with clinician 16, a dental, medical, or pharmaceutical practice that is preauthorized by the patient to receive dental information or prescriptions, an interactive VR/AR kiosk at the office of clinician 16, or any other location that meets with privacy standards set forth by the patient or the family of the patient.

The integration of an interactive VR/AR kiosk provides the capability for patient-facing demonstrations, which may in turn enable a patient to make better-informed decisions with respect to treatment options or treatment packages that are available. In some examples, the patient-facing demonstrations may be integrated into a mobile device application, such as a smartphone app or tablet app, thereby enabling the patient to view demonstrations and prospective treatment outcomes even while the patient is not physically at the dental office.

In this way, system 2 provides dental prescription generation and treatment setup generation while enabling clinician 16 to continue to use his/her hands for patient examination. As such, system 2 provides various technical improvements, such as concurrently providing evaluation/treatment information during an ongoing patient encounter, reducing data errors and/or incompleteness that could be caused by requiring clinician 16 to interrupt treatment/evaluation to then, with some delay, enter data using his/her hands as well as incentivizing error correction by providing clinician 16 subsequent opportunities to review the original treatment proposal for critical evaluation.

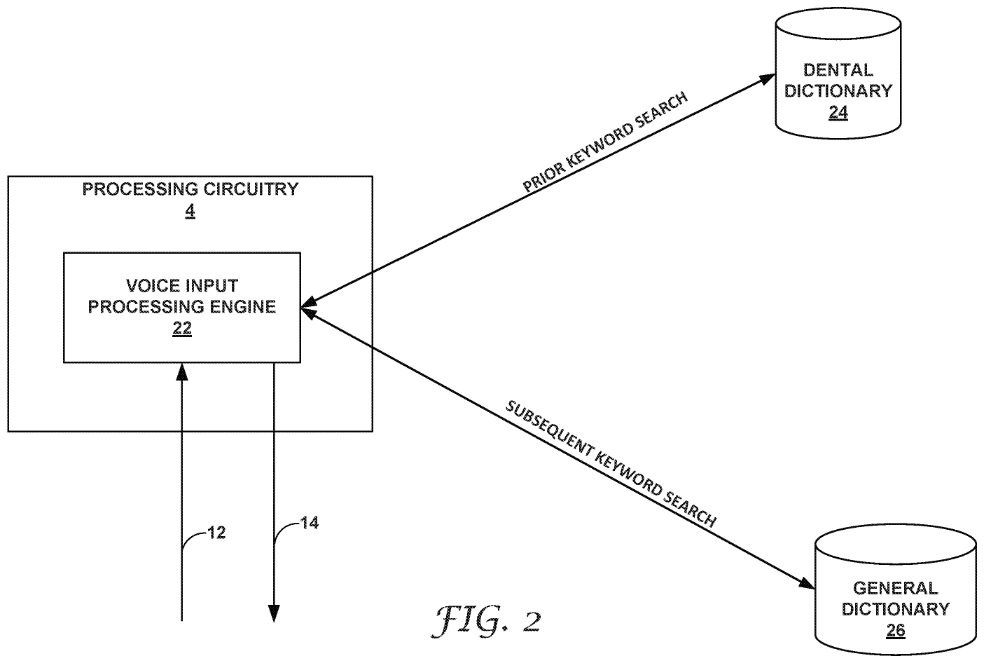

FIG. 2 is a conceptual diagram illustrating dental-oriented natural language processing aspects of this disclosure. FIG. 2 illustrates additional details of custom dental language processing techniques that components of FIG. 1 may perform, in accordance with aspects of this disclosure. In the implementation shown in FIG. 2, processing circuitry 4 includes a voice input processing engine 22. Voice input processing engine 22 receives spoken input data 12, and parses spoken input data 12 for words and phrases. In accordance with some aspects of this disclosure, voice input processing engine 22 leverages the relatively esoteric nature of dental terminology, by using a hierarchical lexicon. For instance, voice input processing engine 22 may analyze spoken input data 12. In some examples, voice input processing engine 22 analyzes spoken input data 12 in a multi-step fashion by using multiple lexicons that selectively prioritize esoteric dental terminology.

That is, voice input processing engine 22 first performs keyword matching of the words/phrases extracted from spoken input data 12 against dental dictionary 24. Dental dictionary 24 represents a targeted collection of words and phrases that have relevance in the fields of general dentistry, orthodontia, oral surgery, maxillofacial surgery, periodontics, endodontics, and prosthodontics. By performing the prior keyword search against the targeted set of words and phrases included in dental dictionary 24, voice input processing engine 22 implements the techniques of this disclosure to focus the keyword analysis of spoken input data 12 on the dental aspects of the spoken input data 12, while initially tuning out the non-dental words, which may represent “noise” with respect to the spoken input provided by clinician 16.

In turn, voice input processing engine 22 may perform a subsequent keyword search using general dictionary 26. For instance, voice input processing engine 22 may collect those words/phrases of the parsed version of spoken input data 12 that did not match any entry of dental dictionary 24, and may compare those words against the entries of general dictionary 26, which represents a lexicon of ordinarily-used words in English or in another language. In some cases, voice input processing engine 22 may use matches in the subsequent keyword search against general dictionary 26 to fill in context in generating dental information 14. For instance, voice input processing engine 22 may use the ordinarily-used words yielded by the subsequent keyword search against general dictionary 26 to link the dental terms yielded by the prior keyword search against dental dictionary 24 to determine actions to be performed, times at which certain actions are to be taken, etc.

In some examples, voice input processing engine 22 may use the subsequent keyword search against general dictionary 26 to extract patient experience information from spoken input data 12. For instance, parts of spoken input data 12 that did not match the esoteric vocabulary reflected in dental dictionary 24 may, at times, represent a conversation between clinician 16 and the patient during the patient encounter. By comparing words and phrases of such conversation against general dictionary 26, voice input processing engine 22 may obtain information reflecting aspects of the patient encounter such as patient comfort, feedback on the surroundings, etc.

Processing circuitry 4 may store analysis of the patient experience to a storage device to be used as heuristic data at a later time, and/or communicate the patient experience information via output devices 10 to guide clinician 16 on possible changes for future patient encounters. By logging patient experience heuristics across multiple patients, in some examples system 2 may automatically formulate practice analytics related to patient experience over time with respect to the entire patient pool or a subset thereof.

In some examples, processing circuitry 4 and/or voice input processing engine 22 may implement machine learning to fine-tune the analysis of spoken input data 12 over time. For instance, processing circuitry 4 may use corrections provided by clinician 16 in the past to update the keyword searching techniques, such as by correcting for pronunciation nuances and other past inaccuracies. In this way, processing circuitry 4 may improve the precision of dental information 14 over time, thereby reducing bandwidth consumption and computing resource usage that might otherwise be expended for corrective measures.

In some examples, the clinician 16 may state a command requesting a prescription summary (e.g., “summarize that prescription to me”), and processing circuitry 4 may cause the system out output an audio restatement of the prescription that the clinician 16 has previously stated. This may allow the clinician to confirm the accuracy and completeness of the prescription. In some examples, the hierarchical arrangement of dental dictionary 24 and general dictionary 26 enable clinician 16 to perform prescription verification. For example, if clinician 16 provides a spoken command such as “summarize that prescription to me,” processing circuitry 4 may perform the first keyword search against dental dictionary 24, and provide a sequence of matching keywords as part of dental information 14. In this way, processing circuitry 4 may avail of the esoteric nature of dental terminology to provide clinician 16 a verification option in which extraneous conversation (noise) is removed from spoken input data 12, enabling clinician 16 to then proceed with further analysis based on prescription-relevant information that was part of the captured conversation.

In this way, voice input processing engine 22 implements the hierarchical keyword searching techniques of this disclosure to more effectively allocate computing resource usage, to reduce computing lag times, and to more efficiently manage the storage devices that implement dental dictionary 24 and general dictionary 26. As one example, dental dictionary 24 may be stored to a faster access, lower capacity device than general dictionary 26. Dental dictionary 24 represents a canonical data set, in that dental dictionary 24 is a condensed (or potentially, minimalized) data set used for voice input processing. Based on the canonical nature of dental dictionary 24, the hierarchical processing techniques of this disclosure improve precision (e.g., by mitigating or potentially eliminating false-positives that may result from homonyms included in a broader lexicon) as well as computing resource usage, by reducing the number of comparisons required to generate a dental state assessment or treatment plan.

In this way, in some examples, processing circuitry may receive voice input data that specifies dental information for a patient, analyze the voice input data to process the dental information, and based on the identified dental treatment information, generate one or more custom dental prescriptions for the patient. In some examples, to analyze the voice input data using natural language processing (NLP) to identify the dental treatment information, processing circuitry 4 may identify a dental dictionary, where the dental dictionary represents a subset of a general NLP dictionary, parse the voice input data to obtain terms, and compare one or more of the terms obtained from the parsed voice input to a portion of the dental dictionary. In some examples, processing circuitry 4 may determine that at least one of the terms obtained from the parsed voice input data does not match any entry of the dental dictionary or the general dictionary, and may discard the at least one term that does not match any entry of the dental dictionary or general dictionary without using the at least one term in generating the custom dental prescription.

In some examples, processing circuitry 4 may determine that at least one of the terms obtained from the parsed voice input data does not match any entry of the dental dictionary, compare the at least one term to a remainder of the general NLP dictionary, where the remainder of the general NLP dictionary does not overlap with the subset represented by the dental dictionary, and based on determining the at least one term matches at least one entry in the remainder of the general NLP dictionary, may include the at least one term in the generated custom dental prescription. In some examples, the voice input data represents, at least in part, a clinician-patient conversation. In these examples, processing circuitry 4 may generate, based on the NLP-based analysis of the voice input data, generating, by the computing device, patient experience information, and may add the patient experience information to a patient experience heuristics repository. In various examples, the dental information includes one or both of dental status information and/or dental treatment information.

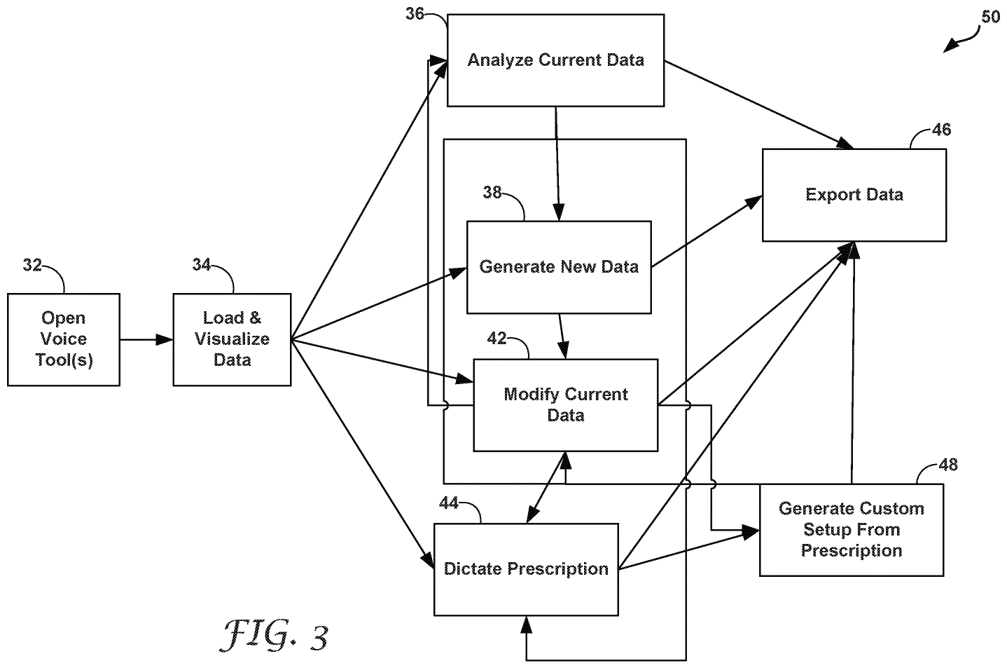

FIG. 3 is a conceptual diagram illustrating an example workflow 50 of disclosure. According to workflow 50 shown in FIG. 3, system 2 enables clinician 16 to dictate a custom oral prescription. Processing circuitry 4 (and optionally processing circuitry of voice input devices 6) may use natural language processing (NLP) and/or various forms of machine learning to analyze the oral prescription represented by voice input data 12. In these examples, processing circuitry 4 may select from a group of final setup generation algorithms, which it can then use to generate a final setup and coordinating intermediate setups for the patient which address the particular needs dictated in the prescription. In one example, the prescription is sent directly to a final setup generator, shown by block 48 titled “generate custom setup from prescription” in FIG. 3 with respect to this particular example. For instance, voice input data 12 may include the phrase “generate setup from prescription,” which voice input processing engine 22 of processing circuitry 4 may parse and analyze (e.g., interpret) to then utilize prescription information for the patient to generate either an intermediate setup or a final setup with respect to orthodontic treatment of the patient.

In another example, processing circuitry 4 may send the prescription to a technician, such as by signaling the prescription using a communications interface (e.g., a wireless or hardwired) network card, over network 8, to a computing device identified with the pertinent technician. The technician, upon checking the prescription and optionally, other requirements, may use the technician's computing device to send the information to a final setup generator. These functionalities are collectively shown by block 46 titled “export data” in FIG. 3. The functionalities described above with respect to block 46 collectively represent the capability of system 2 to submit a case (with respect to the particular patient to whom the prescription pertains) for final setup formation and generation or functionalities enabling clinician 16 to submit a case. Alternatively, in some examples, the technician may use the prescription received from processing circuitry 4 to manually generate the final setup, in addition to or instead of relying on the final setup generator.

According to some examples of this disclosure, system 2 provides clinician 16 with the capability to call up and receive comparison data pertaining to a patient's current state, such as comparison data of the current state as opposed to one or more past dental states of the same patient. According to some examples, system 2 also enables clinician 16 to call up and receive intermediate setups and/or final setup alternatives. These capabilities, collectively, are illustrated in FIG. 3 by way of block 36, titled “analyze current data.” For example, processing circuitry 4 may obtain and analyze patient information in response voice input data 12 including the phrase “tell me about” with respect to a particular tooth or group of teeth. In another example, processing circuitry 4 may obtain and analyze patient information in response voice input data 12 including the phrase “analyze this setup” with respect to an intermediate setup or final setup that has been previously identified via voice input data 12 and is the proposed setup that is currently under discussion. Processing circuitry 4 may implement the call up and retrieval capabilities described with respect to block 36 by identifying a tooth (or group thereof) or a setup (whether intermediate or final) using the functionalities provided by voice input processing engine 22, and extracting the relevant information from a data repository implemented in a storage device, such as one or more hard drives, solid state drives, etc. to which processing circuitry 4 is communicatively coupled.

In accordance with some aspects of this disclosure, system 2 provides clinician 16 the capability to modify a setup by moving individual teeth or groups of teeth. For instance, clinician 16 and others (e.g. an assistant, the patient being examined during the patient encounter, etc.) may discuss individual teeth or groups of teeth. In these scenarios, processing circuitry 4 may use the discussion information captured and relayed in the form of voice input data 12, to generate information based on a planned modification of the position(s) of the tooth or grouping thereof. According to these examples, system 2 enables clinician 16 (and optionally, others) to modify the position of a tooth or a group of teeth in a particular arch by utilizing any of the Dental Universal Numbering System, the Palmer Notation Numbering System, the Federation Dentaire Internationale Numbering System, or an alphanumeric tooth numbering system to identify the tooth/teeth being discussed for modification. These capabilities are illustrated in FIG. 3 in block 42, which is titled “modify current data.” Some specific non-limiting examples of modification commands that processing circuitry 4 may process according to functionalities described with respect to block 42 are “torque tooth [tooth number] distally,” “torque tooth [tooth number] proximally,” “expand” (e.g., “expand an arch” or as otherwise applied to a group of teeth or inter-tooth spacing), “optimize,” etc.

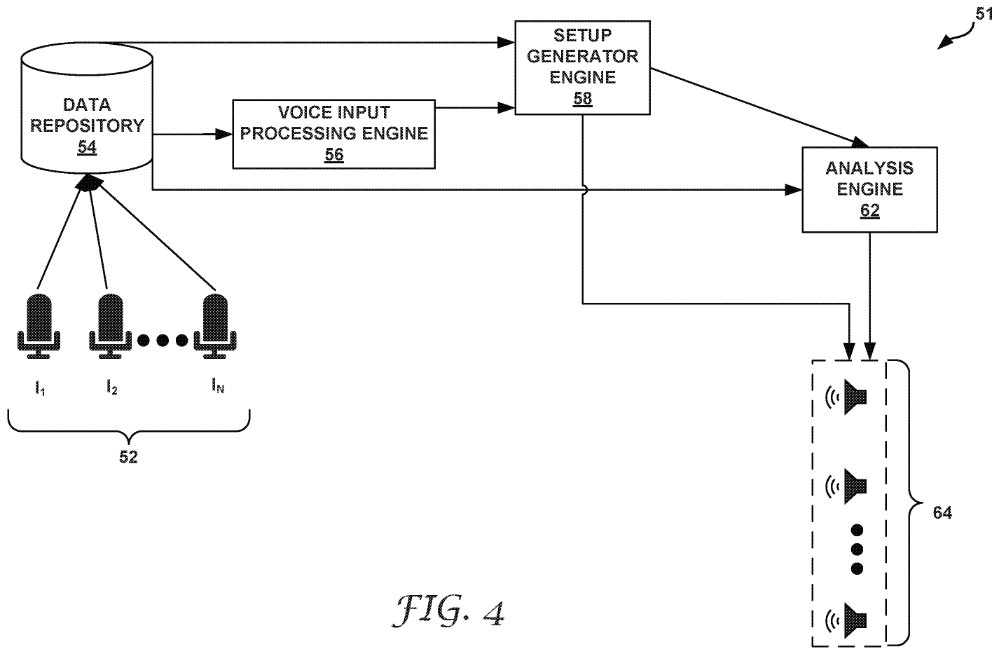

FIG. 4 is a block diagram illustrating a system 51, which is one example implementation of system 2 illustrated in FIG. 1. According to one particular configuration of system 51, clinician 16 may use locally-provided voice inputs to instruct a cloud-based system to retrieve, process, generate, or analyze an orthodontic configuration. As such, system 51 of FIG. 4 provides clinician 16 with results locally, via web-based computing tools (such as cloud computing software, e.g., Azure® by Microsoft® Corporation, Amazon Web Services (AWS®) by Amazon® Inc, etc.).

In the example of system 51 of FIG. 4, a series of voice inputs I_{I} through I _{N} (collectively, voice inputs 52) are provided from one or more locations (e.g., a dental examination room, a dentist's private office, etc.) to a data repository 54. Data repository 54 may be implemented locally, remotely, or in a distributed fashion over multiple locations, in accordance with the system configurations of this disclosure. For purposes of discussion, system 51 is described herein with respect to data repository 54 being implemented remotely, and as such, voice inputs 52 are described as being provided to data repository 54 over a network connection, such as a connection to network 8 of FIG. 1. In this way, system 51 is described herein as a cloud-based system, although the cloud-based infrastructure is not a requirement for the techniques of this disclosure to be performed.

One or both of NLP engine 56 and/or setup generator engine 58 may obtain portions of voice inputs 52 from data repository 54. For instance, NLP engine 56 and/or setup generator engine 58 may demarcate individual inputs from one another, based on various criteria, such as the length of time between respective timestamps, differences between speakers (e.g., as discerned using voice recognition of known and/or unknown users), and various other criteria. With respect to a particular single input or finite set of inputs obtained from data repository 46, NLP engine 56 may parse individual words and phrases (collectively, “tokens”) and compare the tokens to different lexicons in a preferential order. For instance, NLP engine 56 may first compare the tokens against a specialized dental dictionary, and then compare tokens that did not yield a match in the first search against a general dictionary. Various custom dental NLP techniques that NLP engine 56 is configured to implement according to aspects of this disclosure are described above with respect to voice input processing engine 22 of FIG. 2 and are not described in detail again with respect to NLP engine 56.

Setup generator engine 58 may implement various techniques of this disclosure (whether rooted in NLP, other forms of machine learning, or otherwise), to generate an orthodontic setup, such as an intermediate setup or a final setup, using portions of voice inputs 52 obtained from data repository 54. In various examples, setup generator engine 58 may obtain raw data directly from data repository 54, parsed and processed data indirectly from repository 54 via NLP engine 56, or combinations thereof. In any event, setup generator engine 58 may form orthodontic setups for a particular intermediate stage or the projected final outcome of a particular patient's orthodontic treatment.

Although illustrated as two separate components, it will be understood that NLP engine 56 and setup generator engine may, in various examples, overlap partially or may be integrated into a single hardware unit. Whether implemented separately, or integrated partially or fully, NLP engine 56 and setup generator engine may be implemented in one or more of programmable processing circuitry, fixed function circuitry, one or more processors, such as one or more digital signal processors (DSPs), general purpose microprocessors, application specific integrated circuits (ASICs), field programmable gate arrays (FPGAs), or other equivalent integrated or discrete logic circuitry, as well as any combination of such components.

Analysis engine 62 may obtain the processed data that are output by setup generator engine 58 and/or NLP engine 56, and optionally, may obtain additional portions of raw data representing voice inputs 52 from data repository 54. Analysis engine 62 may perform end-stage analysis of the orthodontic setup obtained from setup generator engine 58, the processed prescription or evaluation data obtained from NLP engine 52, and/or portions of raw data obtained from data repository 54 to generate a refined analysis of voice inputs 52. As an example, analysis engine 62 may obtain contextual information directly from data repository 54 to discern patient comfort information or other surrounding information describing aspects of the patient encounter(s).

In turn, analysis engine 62 may provide dental information (which represents a result of the end-stage analysis) as outputs 64 to output devices, such as output devices deployed at the dental office where the patient encounter is presently occurring or occurred in the past. Moreover, as shown in FIG. 4, setup generator engine 58 may directly provide a portion or all of outputs 64, as well. For instance, setup generator engine 58 may provide a description of an intermediate or final setup as part of outputs 64. Outputs 64 may represent data that is expressed in human-intelligible form by various output devices, such as in the form of audio output via one or more speakers (shown as an example in FIG. 4) or headphones, or in readable form or illustrated form via one or more display devices, or in any other form that is intelligible to clinician 16 or others.

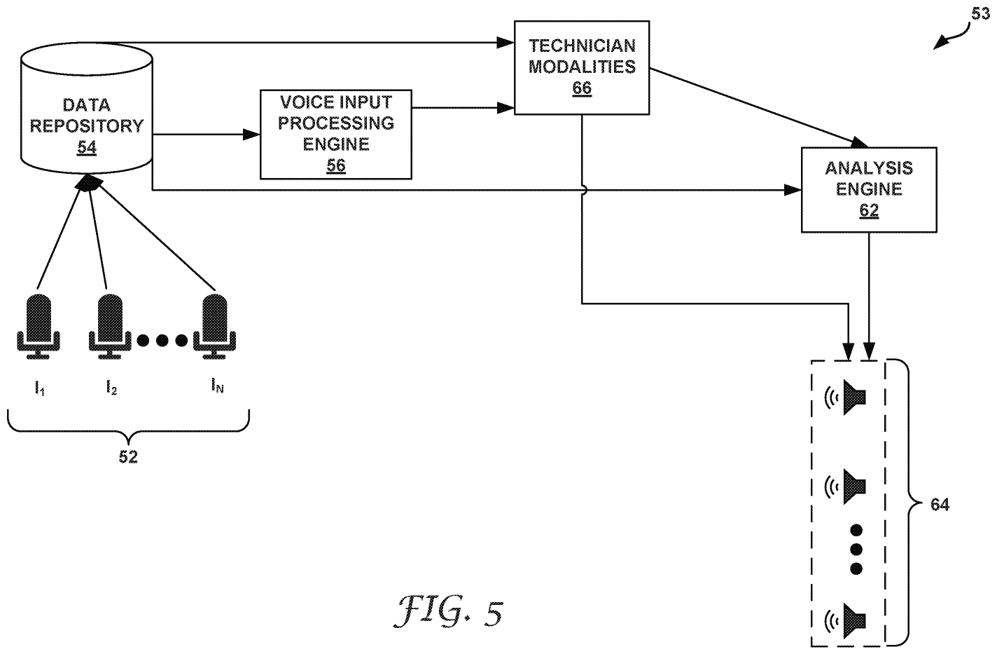

FIG. 5 is a block diagram illustrating a system 53, which is another example implementation of system 2 illustrated in FIG. 1. System 53 includes several elements that are numbered similarly to elements of system 51 illustrated in FIG. 4. Similarly-numbered elements that were described above with respect to FIG. 4 are not described separately with respect to FIG. 5, for the purpose of brevity.

System 53 represents an implementation in which orthodontic setups are generated manually by one or more technicians, rather than by the setup generator engine 58 of FIG. 4. In the example of system 53 of FIG. 5, various technician modalities (technician modalities 66) obtain parsed and analyzed information from NLP engine 56 and/or raw data from data repository 54.

Technician modalities 66 represent computing hardware, including network-connected computing devices, that are capable of obtaining data from NLP engine 56 and/or data repository 54, and present the data in intelligible form to the technician(s). Technician modalities 66 may include various types of devices, including general-purpose hardware such as tablet computers, smartphones, PDAs, laptop/notebook/netbook computers, desktop computers, and/or specialized hardware such as medical computers, ruggedized touchscreen devices that process dental codes, etc.

In the example of FIG. 5, the technician(s) operating technician modalities 66 may provide the setup information to analysis engine 62 and/or to the devices that process and present outputs 64. In this way, as illustrated in FIGS. 4 and 5, the systems of this disclosure are compatible with both automated orthodontic setup generation and manual orthodontic setup generation, while supporting both while limiting the need to update hardware and communication infrastructures.

In either of systems 51 or 53, whether setups are human-generated (using technician modalities 66) or machine-generated (by setup generator engine 58), systems 51 and 53 provide clinician 16 with interchangeable, and sometimes identical, user experiences. Described in the context of either FIG. 4 or FIG. 5, systems 51 and/or 53 may leverage voice inputs 52 to perform various functionalities related to the dental evaluation or treatment of a patient. As non-limiting examples, components of systems 51 and/or 53 may leverage voice inputs 52 to receive patient identification data, to upload dental scans, to upload photos of the patient, or to receive prescription data, to receive data on tooth position, landmark, etc. As additional non-limiting examples, components of systems 51 and/or 53 may leverage voice inputs 52 to receive data on tooth position modifications, to receive data on launching final and intermediate setup generation, or to receive data queries from clinician 16. An example of patient identification functionality is the capability to search patient records based on a unique identifier to select the particular patient to whom subsequent voice inputs 52 of the patient encounter are applicable.

As described above, communication hardware of systems 51 and/or 53 may send voice inputs 52 to data repository 54. Although data repository 54 is described as representing cloud-based storage in the context of FIGS. 4 and 5, in other examples data repository 54 may be positioned in a variety of physical locations in accordance with aspects of this disclosure. As such, in the cloud storage implementation described herein, the communication hardware of systems 51 and/or 53 are configured to signal digital data representing voice inputs 52 to data repository 54 over a network connection, such as a VPN or a public network such as the Internet.

Data repository 54 may store various types of data, including the digital data received from voice inputs 52, as well as final setups and/or intermediate setups data determined for patients, treatment time data for various patients, treatment cost data for the patients, etc. The various stored data may apply to one or more patients, such as a patient of the current patient encounter, as well as past or subsequently-examined patient(s). Various components illustrated in FIGS. 4 and 5 may obtain data from data repository 54, whether via local connections or via network connectivity. Such components include any one or more of NLP engine 56, setup generator 58, technician modalities 66, or analysis engine 62.

NLP engine 56 may receive data from the cloud-based storage represented by data repository 54 and/or from a local tracker. NLP engine 56 is configured to run natural language processing algorithms on the received data for various end uses, such as to assist in the generation of requirements for final and intermediate setups. For instance, NLP engine 56 may send the requirements to setup generator engine 58 and/or to send the requirements to technician modalities 66. Again, the communication functionalities attributed to NLP engine 56 above may be local, may be cloud-based, or a combination thereof.

Setup generator engine 58 of system 51 may also receive patient data from data repository 54, and/or to receive requirements data from NLP engine 56. Further, setup generator engine 58 may also store one or multiple algorithms for generating final and intermediate setups to one or more storage hardware components to which setup generator engine 58 is communicatively coupled. Setup generator engine 58 may be configured to generate final setups and/or intermediate setups based on the patient data and requirements data obtained from data repository 54 and NLP engine 56, respectively. In turn, setup generator 58 may send final setups and/or intermediate setups to analysis engine 62 and to various auditory and/or visual output devices as outputs 64. Again, the communication functionalities attributed to setup generator engine 58 above may be local, may be cloud-based, or a combination thereof.

Analysis engine 62 may receive data from data repository 54 and/or from one or more local trackers deployed at the location where the relevant patient encounter occurred or is currently occurring. Analysis engine is configured to run evaluation functions. As non-limiting examples, analysis engine 62 may be configured to compute comparisons between different final setups and/or between intermediate setups and/or between combinations thereof. Analysis engine 62 may also be configured to generate descriptive and/or inferential statistics, to compute an anticipated treatment time for a patient, or to compute anticipated costs for treatment of a patient. As shown in FIGS. 4 and 5, analysis engine 62 may also send results of the various analyses/functions, in the form of outputs 64, to auditory and/or visual output devices. Again, the communication functionalities attributed to analysis engine 62 above may be local, may be cloud-based, or a combination thereof.

Outputs 64 may include auditory and/or visual output data and may be output locally at a location of a current or past patient encounter to which outputs 64 are tied. Outputs 64 may include, in various examples, displays of malocclusion, final setups, intermediate setups, etc. In some examples, outputs 64 may convey indicators of severity of malocclusion, qualities of final setups and/or intermediate setups, and may include one or both of visual and/or auditory data. Examples of such indicators/qualities may include one or more of metrics, teeth of interest, ranking, pass/fail statuses, heat maps, flags, etc. In some examples, the components illustrated in FIGS. 4 and 5 may, whether individually or acting in concert, update outputs 64 in real time or in response to user input (e.g., voice inputs 52 and/or other input data).

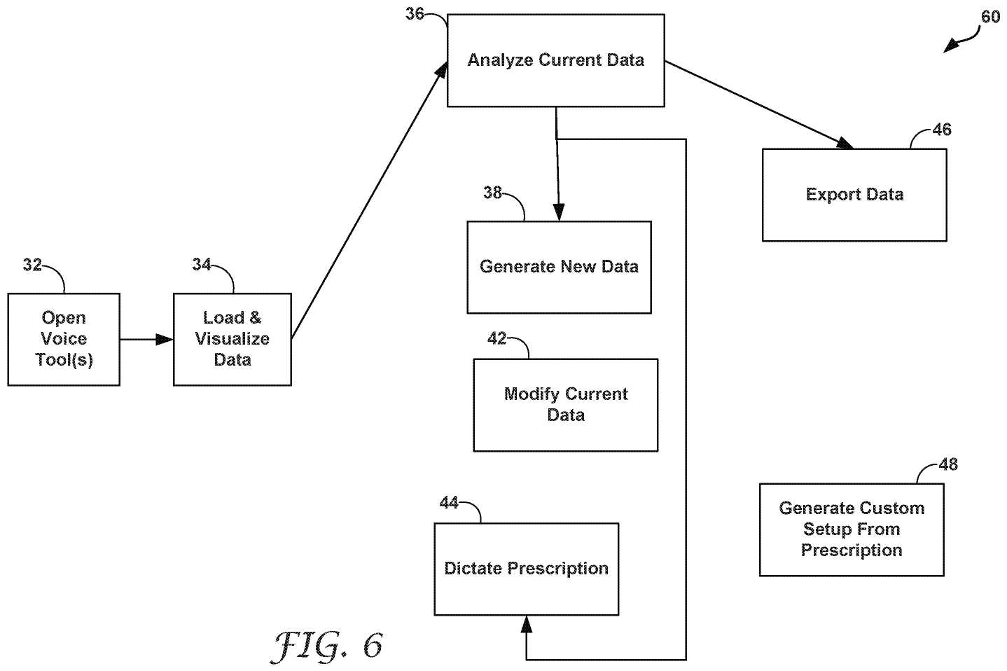

FIG. 6 is a conceptual diagram illustrating an example workflow 60 of disclosure. Workflow 60 is a variation of workflow 50 illustrated in FIG. 3 and represents an example of a workflow originating with an analysis of a malocclusion or setup. Workflow 60 represents a process that may be executed partially or wholly by a virtual assistant or by another device or set of devices configured to perform the techniques of this disclosure. In the example of workflow 60, after using voice commands to open a user interface and load patient data, clinician 16 may interact with the system to analyze patient scans. For instance, clinician 16 may leverage the analysis capabilities shown in block 36 to learn more about the patient data. As examples, clinician 16 may leverage the data analysis capabilities shown in block 36 to learn about metrics or orthodontic scores to determine whether the metrics/scores are within acceptable ranges, or to identify one or more teeth in need of improvement.

For instance, by identifying one or more teeth that are below a threshold on a score or on the basis of a particular orthodontic treatment criterion (whether preset or set by clinician 16), clinician 16 may determine an equivalent American Board of Orthodontics (ABO) score for the final/intermediate setup currently under analysis or may determine other analytical measures thereof. Utilizing this data, clinician 16 may either export the scan as is (i.e., progressing to the functionalities represented by block 46 in workflow 60), or may invoke a selected one from a set of final setup algorithms to generate a final setup (provided that the data under analysis is related to one or more malocclusions). In some instances, clinician may use the updated understanding of the patient data to dictate a prescription for the patient (i.e. progressing to the functionalities represented by block 44 in workflow 60).

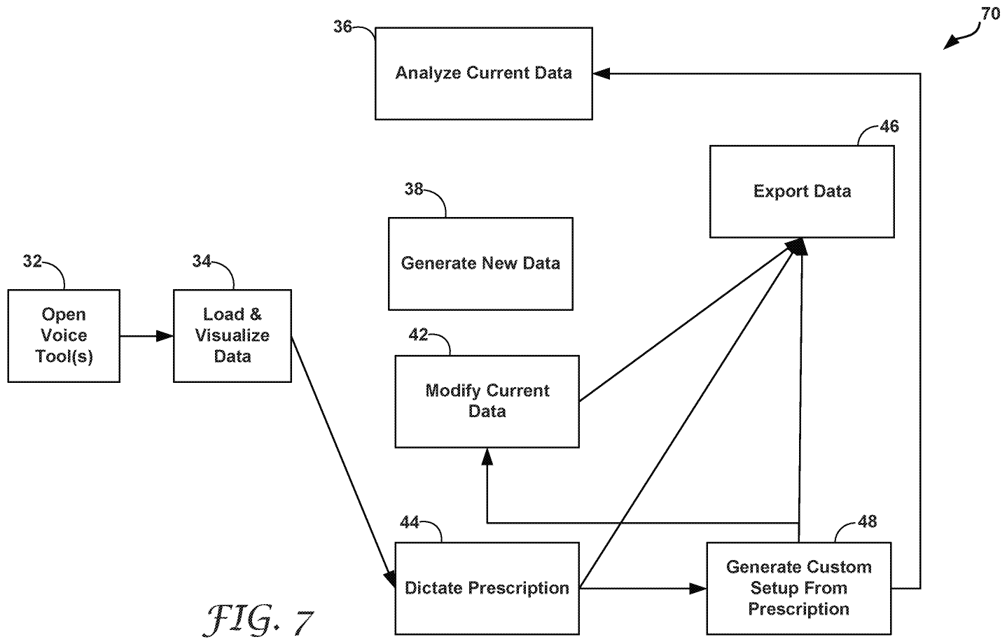

FIG. 7 is a conceptual diagram illustrating an example workflow 70 of disclosure. Workflow 70 is a variation of workflow 50 illustrated in FIG. 3, and represents a workflow originating with a dictated prescription. In the example of workflow 70, either after execution of the analysis functionalities (block 36) or immediately after loading patient data such as a scan (represented by the functionalities of block 34), clinician 16 may dictate treatment preferences by providing voice input data 12, e.g. to a virtual assistant device. For example, upon receiving a prescription-introducing command such as “record prescription,” the virtual assistant may transition into an extended listening mode. While functioning in the extended listening mode, the virtual assistant may continue recording for a certain period of time, not stopping during relatively longer thought-pauses. That is, the virtual assistant configured according to user-facing aspects of this disclosure may accommodate various stoppages in speech that occur during prescription dictation, rather than defaulting to a determination that the command has ended and exiting a program or speech-capture action.

Instead, the virtual assistant may detect the end of a dictated prescription using a custom ending command that is unlikely to be spoken accidentally. In one example where the virtual assistant implements user-facing capabilities of this disclosure by building on a platform of Amazon® Inc's Alexa® suite, the virtual assistant may detect the end of a dictated prescription upon receiving the spoken command “Alexa, end my prescription.” That is, in response to receiving the predetermined custom ending command, the virtual assistant may cease to operate in the extended listening, and may terminate recording, or at least temporarily suspend recording, pending receipt of a custom start command.

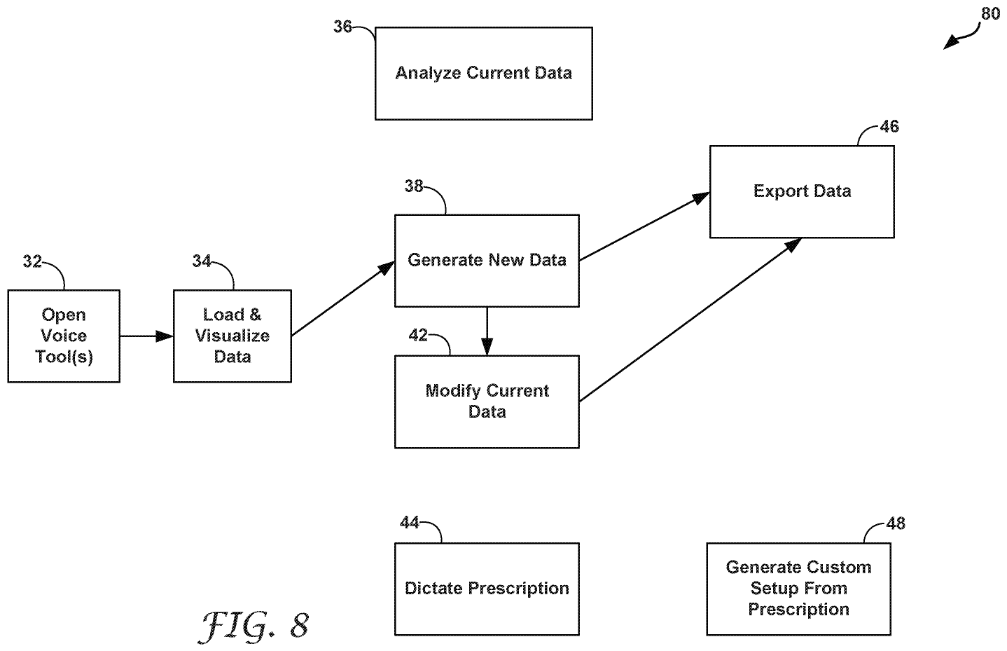

According to workflow 70, the virtual assistant may save the audio data as one or more of an audio file (such as in .mp4 format or other audio formats), and/or as a transcribed text file (e.g., txt format, etc.). For instance, the virtual assistant may store the transcribed text file to data repository 54 for submission to NLP engine 56. NLP engine 56 may analyze the prescription represented by the transcription for key text. In these and other examples, setup generator 58 may identify a custom final setup generation algorithm from a set of possibilities and may generates a potential final setup chosen based on the analysis of the spoken prescription (shown by way of the functionalities of block 48). If clinician 16 declines to dictate a prescription and instead selects one of a present grouping of final setup algorithms, such as by using a preset name, as described below in greater detail with respect to FIG. 8. This final setup can then be modified (e.g., using functionalities attributed to block 42), analyzed (e.g., using functionalities described with respect to block 36), submitted directly to a technician for refinement along with the text and/or audio prescription, or submitted directly for orthodontia to be created (e.g., according to functionalities attributed to block 48). Orthodontia that might be created in this fashion may be in the form of clear tray aligners, in the form of brackets and wires, in the form of custom lingual brackets, etc.

FIG. 8 is a conceptual diagram illustrating an example workflow 80 of disclosure. Workflow 80 is a variation of workflow 50 illustrated in FIG. 3 and represents an example in which the workflow originates with setup generation. In the example of workflow 80, clinician 16 can proceed directly to setup generation (represented by functionalities attributed to block 48), or can proceed to setup generation after an analysis or prescription transcription is performed, in accordance with the descriptions above of workflows 60 and 70. By proceeding directly to setup generation (block 48), clinician 16 may call one of a set of preloaded final setup generation algorithms and/or intermediate setup generation algorithms, and may produce the setup results for interaction on a visual display of output devices 10.

According to functionalities attributed to block 48, a number of algorithms are accessible to clinician 16 for selection via the virtual assistant device. Examples include “Final Setups Social Six,” “Final Setups Spacing Only,” “Final Setups Full Setup,” “Intermediate Setups From This Final,” and others. Upon viewing the resulting setups, clinician 16 can either submit the data to technicians for refinement or submit the data for orthodontia creation. In some examples, clinician 16 may proceed to a modification step (e.g. as shown by way of block 42) in which clinician 16 can tweak the result himself/herself via input devices 6. Analysis and dictation steps (shown in blocks 36 and 42, respectively) after reviewing the new final setup are also possible within the scope of workflow 80 and are discussed above with respect to workflows 60 and 70.

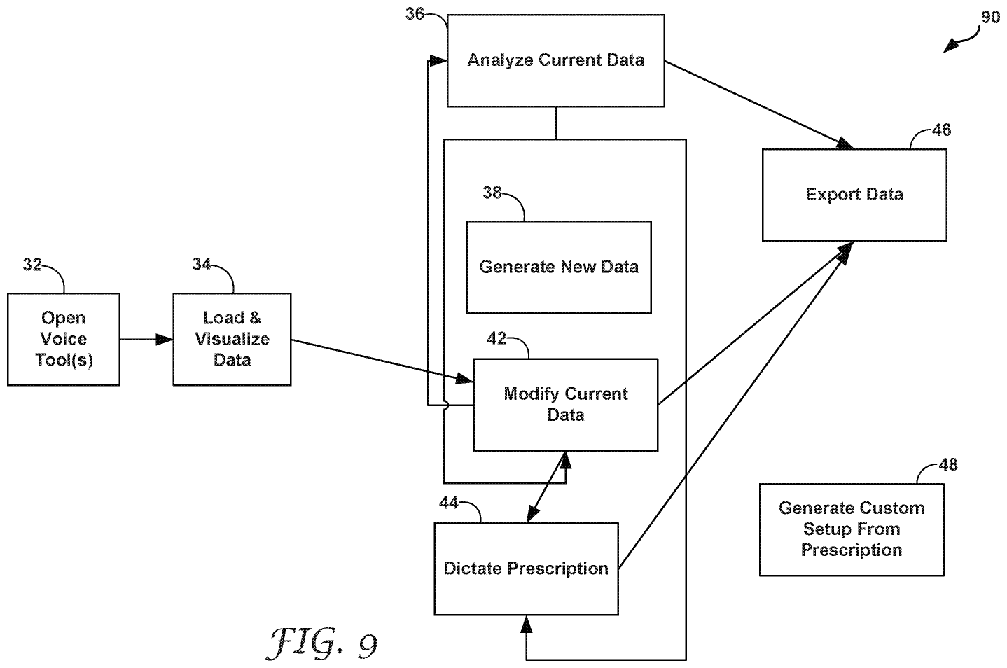

FIG. 9 is a conceptual diagram illustrating an example workflow 90 of disclosure. Workflow 90 is a variation of workflow 50 illustrated in FIG. 3 and represents an example in which the workflow originates with modification of a previously-loaded setup or with a malocclusion. In the modification step (provided via functionalities attributed to block 42), clinician 16 can interact vocally with the virtual assistant to modify the loaded setup, while interacting with a visual display using a different type of input device of input devices 6, such as a mouse. According to workflow 90, clinician 16 can perform actions that modify the entire arch of a scan (e.g. using commands such as “optimize,” “expand,” etc.), actions that modify a grouping of teeth (e.g. “level social six,” “close spaces on upper molars,” etc.), or actions that modify a single tooth (e.g. “torque [tooth number] five degrees,” “extrude [tooth number] two millimeters,” etc.). By identifying commonly-used orthodontic actions, such as “expand,” “extrude,” “intrude,” etc., systems 2, 51, and 53 may create a library of common commands to be used for analyzing and interpreting spoken commands provided by clinician 16.

Clinician 16 may also invoke analysis (by way of functionalities represented by block 36) to be run after the modification step (provided by the functionalities of block 42), or may initiate a loop between modification, analysis, further modification, further analysis, until a desired result is achieved. Upon achieving a setup that represents or is sufficiently similar to the desired result, clinician 16 may export the data to a technician for refinement, or to a third party provider for orthodontia generation (both of which are provided by the functionalities attributed to block 46). Alternatively, or in addition, clinician 16 may proceed to a dictation step (provided by the functionalities described with respect to block 44) to add treatment preferences for the technicians to reference. According to workflow 90, clinician 16 could potentially use a mixed mode of treatment planning wherein clinician 16 modifies particular teeth to suit preferences, and have a technician create the remainder of the treatment plan while leaving fixed the teeth specified by clinician 16 for specific modifications.

Workflows 50, 60, 70, 80 and 90 provide several ways in which clinician 16 can interact with any of systems 2, 51, or 53 to load or view scan data, to access and use treatment planning algorithms, and utilize analysis algorithms in combinatorial profusion. That is, according to techniques and system configurations of this disclosure, clinician 16 can cause these various elements to interact with each other to produce a versatile orthodontic assistant, in order to aid clinician 16 in better understanding patients' scans and to more effectively generate setups or treatment preferences suited to the individual style or idiosyncratic needs of each patient.

According to some aspects of this disclosure, systems 2, 51, or 53 may enable the deployment of a device in an orthodontic waiting room with a virtual assistant, display, and camera with sufficient resolution that would enable patients to take pictures of themselves (e.g., with an open mouth smile or in another pose that exposes some of the patient's teeth), and then run a smile simulator tool. As examples, the deployed devices may incorporate VR/AR capabilities, and may take the form of headsets or six-surface displays. Such systems may run (locally or over the cloud) a simplified final setup generation algorithm, extract the image of the patient's teeth from the remainder of the picture, register the new orthodontic setup against the patients face and lips, and show the patient what the patient could potentially look like after a particular treatment plan is administered. With the implementation of certain algorithms, an AR display may display, to the patient, the patient's own face with the new prospective dentition substituted in or superimposed, thereby providing a “virtual mirror” that presents post-treatment projections of the patient's appearance. Patient buy-ins may be incentivized with the addition of such tools at a dentist's office.

Additionally, in some examples, if the algorithms underlying such a smile simulator are not fast enough for real-time use, the systems of this disclosure may still enable the patient to interact with the simulator interface at check-in. For instance, the patient could have his/her picture taken, and the systems of this disclosure may run the algorithms in the background during the patient encounter and/or waiting time. The systems of this disclosure may then display the prospective appearance via facial image modification at the end of the appointment or later, such as by emailing the images to the patient.

In some such patient interaction aspects of this disclosure, the systems described herein may enable the patients to interact with the system and thereby learn about the various treatment options available to the patients. As examples, the patients may ask and answer queries related to treatment speed or timelines, aesthetics, costs, maintenance needs, etc., and may thereby obtain a suitable treatment plan. Examples of patient-provided queries include, but are not limited to, “how will I look wearing aligners?” or “how will I look wearing braces?” In turn, the systems of this disclosure may provide responses with the corresponding projected facial image or VR/AR visual output. As another example, a patient can also ask “how will I look after treatment?” to see the post-treatment smile simulation.

According to some examples compatible with the systems of this disclosure, devices such as a dental hygienist's assistance device, or a dental assistant's virtual assistance device record oral data during periodic (e.g., biyearly) or ad-hoc cleanings, while the hygienist or dental assistant has his/her hands in the patient's mouth. For example, when using a probe to test pocket depths along the gum line, hygienists typically call out the pocket depths and then must remember these numbers when they remove their hands from the patient's mouth. The systems of this disclosure may enable the hygienist to probe the patient, normally speaking the numbers out loud for recording and further application/analysis, without requiring the hygienist to commit the numbers to memory, without having to put down the dental examination equipment, and without having to remove gloves, type on a computer, put on fresh gloves, and return to the examination. These systems of this disclosure may also cut down on the need to have multiple hygienists and/or scribes and/or dental assistants in the room during a patient encounter. In this way, the systems of this disclosure provide potential advantages with respect to several aspects of dental treatment, such as improved efficiency and hygiene (e.g., by cutting the risk of infections introduced by interruptions that cause hygienists to touch computing equipment during evaluation), etc.

For instance, the user-facing interfaces of this disclosure may load a gumline annotation screen and automatically fill in the gingival pocket depths as they are spoken aloud, allowing the hygienists to save time, effort, and/or material (gloves), and reducing a potential source of contamination between hands and patients' mouths. Other patient findings may be recorded and transcribed for a dentist when the dentist enters the room for consultation, such as unusual bleeding, gingival sensitivity, pre-cavity formation(s), etc. Due to the nature of their jobs, hygienists cannot type or control a computer by hand for a significant amount of time taken for a patient encounter, and as such, the speech-operated dental assistant technology of this disclosure serves as a time-saving and effort-saving system during the average workday of these busy professionals.

According to some examples of this disclosure, clinician 16 (whether a dentist, technician, orthodontic assistant, or any other treatment coordinator) may, at any point in time, ask about an order identification or case. Non-limiting examples of these voice inputs include “What's the status of my order?,” “When is my next appointment with the patient?,” “Has the treatment plan been received from [external provider name]?,” or “Show me the treatment plan?” Patient progress monitoring may also be a incorporated into these configurations, by enabling clinician 16 to ask “How is the patient tracking?” to engage a progress monitoring routine that would enable analysis of the patient's orthodontic treatment progress. Systems of this disclosure may make some of this information available in a patient facing app, such as an app that enables patients to learn the status of their orthodontia post ordering (e.g. “Trays being made”, “Shipped”, “Arrival expected tomorrow”), or self-track the patient's own progress with treatment. According to some examples of this disclosure, the systems described herein may enable users to control a three-dimensional (3D) viewer via voice commands. For instance, the orthodontist or technicians would be able to issue commands such as “Show me occlusal view,” “show me front view,” “hide lower arch,” “make clinical notes,” etc. These configurations may enable clinician 16 to annotate treatment or health issues about specific teeth. Moreover, as described above, the systems of this disclosure may enable clinician 16 to identify a case or filter a set of cases with commands such as “Show me all patients I saw last week,” or “Open a case for [patient identifying information].” These capabilities provided by the systems of this disclosure represent portal integration, in that the systems provide clinician 16 with the ability to access and/or edit patient records within a dental records system, view the status of orders via ordering systems, etc.

In some examples, this disclosure is directed to a method that includes receiving, by a computing device, voice input data specifying a patient and dental scan information associated with the patient, and responsive to receiving the voice input, loading, by the computing device, the dental scan associated with the patient specified in the voice input data. In some examples, this disclosure is directed to a method that includes receiving, by a computing device, voice input data specifying at least information identifying a patient and a command, identifying, by the computing device and based on the command, a predetermined dental treatment setup, and assigning, by the computing device, the identified predetermined dental treatment setup to the patient. In some examples, this disclosure is directed to a method that includes receiving, by a computing device, voice input data specifying at least information identifying a patient and a command, and comparing, by the computing device and based on the voice input data, a current dental state of the patient to a prior dental state of the patient. In some examples, this disclosure is directed to a method that includes receiving, by a computing device, voice input data, and selecting, by the computing device, based on data of the voice input data, a numbering system from a plurality of available numbering systems to be used for assessing a dental state of a patient. A numbering system as used herein represents a dental numbering system or dental notation that is used to identify individual teeth or a group of teeth. A numbering system as used herein may use a number-based nomenclature or a nomenclature that uses numbers in combination with letters to identify a tooth or a group of teeth.

In another example, this disclosure is directed to a method that includes receiving, by a computing device, voice input data specifying at least information identifying a patient and a command, identifying, by the computing device and based on the command, dental state information associated with the patient, and outputting, by the computing device, the dental state information associated with the patient.

In another example, this disclosure is directed to a method that includes receiving, by a computing device, voice input data specifying at least information identifying a patient and a command, comparing, by the computing device and based on the command, dental state information associated with the patient against dental state information for a population of patients to obtain comparative dental state information associated with the patient, and outputting, by the computing device, the comparative dental state information associated with the patient.

Devices and systems of this disclosure may include, in addition to processors or processing circuitry, various types of memory. Memory devices or components of this disclosure may include a computer-readable storage medium or computer-readable storage device. In some examples, the memory includes one or more of a short-term memory or a long-term memory. The memory may include, for example, RAM, DRAM, SRAM, magnetic discs, optical discs, flash memories, or forms of EPROM, or EEPROM. In some examples, the memory is used to store program instructions for execution by processors or processing circuitry communicatively coupled thereto. The memory may be used by software or applications running on various devices or systems to temporarily store information during program execution.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory

(RAM) such as synchronous dynamic random access memory (SDRAM), read-only memory (ROM), nonvolatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor”, as used may refer to any of the foregoing structure or any other structure suitable for implementation of the techniques described. In addition, in some aspects, the functionality described may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set).

Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially.

In some examples, a computer-readable storage medium includes a non-transitory medium. The term “non-transitory” indicates, in some examples, that the storage medium is not embodied in a carrier wave or a propagated signal. In certain examples, a non-transitory storage medium stores data that can, over time, change (e.g., in RAM or cache).

Various examples have been described. These and other examples are within the scope of the following claims.

1. A method comprising:
receiving, by a computing device, voice input data that specifies dental information for a patient;
analyzing, by the computing device, the voice input data to process the dental information; and
based on the identified dental treatment information, generating, by the computing device, one or more custom dental prescriptions for the patient.

2. The method of claim 1, wherein analyzing the voice input data using natural language processing (NLP) to identify the dental treatment information comprises:
identifying a dental dictionary, wherein the dental dictionary represents a subset of a general NLP dictionary;
parsing the voice input data to obtain terms; and
comparing one or more of the terms obtained from the parsed voice input to a portion of the dental dictionary.

3. The method of claim 2, further comprising:
determining that at least one of the terms obtained from the parsed voice input data does not match any entry of the dental dictionary or the general dictionary; and
discarding the at least one term that does not match any entry of the dental dictionary without using the at least one term in generating the custom dental prescription.

4. The method of claim 2, further comprising:
determining that at least one of the terms obtained from the parsed voice input data does not match any entry of the dental dictionary;
comparing the at least one term to a remainder of the general NLP dictionary, wherein the remainder of the general NLP dictionary does not overlap with the subset represented by the dental dictionary; and
based on determining the at least one term matches at least one entry in the remainder of the general NLP dictionary, including the at least one term in the generated custom dental prescription.

5. The method of claim 1, wherein the voice input data represents, at least in part, a clinician-patient conversation, the method further comprising:
based on the NLP-based analysis of the voice input data, generating, by the computing device, patient experience information; and
adding the patient experience information to a patient experience heuristics repository.

6. The method of claim 1, wherein the dental information includes one or both of dental status information or dental treatment information.

7-10. (canceled)

11. A method comprising:
receiving, by a computing device, voice input data specifying a patient and information for modifying a configuration of a tooth or a group of teeth of the patient;
identifying, by the computing device and based on the voice input data, stored data associated with the tooth or group of teeth of a patient; and
generating, by the computing device and based on the voice input and the stored data, one or more dental arrangements in which the configuration of the group of teeth is modified according to the information for modifying the configuration.

12. The method of claim 11, further comprising:
outputting, by the computing device and for display, a visual representation of the original dental arrangement and any of the one or more dental arrangements in which the tooth or group of teeth is moved.

13. The method of claim 12, wherein receiving the voice input comprises receiving the voice input from a computer-mediated reality interface, and wherein outputting the visual representation of the prospective dental scan comprises outputting the visual representation of the prospective dental scan to display hardware of the computer-mediated reality interface.

14. The method of claim 13, wherein the computer-mediated reality interface comprises one of a virtual reality (VR) interface or an augmented reality (AR) interface.

15. The method of claim 11, wherein the group of teeth includes two or more teeth, and wherein generating the prospective dental scan comprises generating data representing a synergistic movement of all of the two or more teeth included in the group.

16. The method of claim 11, further comprising:
analyzing the voice input data using natural language processing (NLP) to obtain information for modifying a configuration of a group of teeth, at least in part by:
identifying a dental dictionary, wherein the dental dictionary represents a subset of a general NLP dictionary;
parsing the voice input data to obtain terms; and
comparing one or more of the terms obtained from the parsed voice input data to a portion of the dental dictionary.

17. The method of claim 16, further comprising:
determining that at least one of the terms obtained from the parsed voice input does not match any entry of the dental dictionary; and
discarding the at least one term that does not match any entry of the dental dictionary without using the at least one term in generating the one or more dental arrangements.

18. The method of claim 11, further comprising:
outputting, by the computing device and for display, a visual representation of the original dental arrangement.

19-20. (canceled)
